(19) United States
(12) Patent Application Publication
(10) Pub. No.: US 2026/0367057 A1
(43) Pub. Date: Nov. 19, 2026

(54) Monitoring Plant Health During Area Coverage

(71) Applicant: Autonomous Solutions, Inc., Mendon, UT (US)

(72) Inventor: Taylor Bybee, Mendon, UT (US)

(73) Assignee: Autonomous Solutions, Inc., Mendon, UT (US)

(21) Appl. No.: 19/529,046

(22) Filed: Feb. 3, 2026

Related U.S. Application Data
(60) Provisional application No. 63/753,931, filed on Feb. 4, 2025.

(51) Int. Cl. G01N 33/00; A01G 7/00; G05D 1/648; G05D 1/65; G05D 105/15; G05D 105/80

(52) U.S. Cl. CPC G01N 33/0098; A01G 7/00; G05D 1/648; G05D 1/65; G05D 2105/15; G05D 2105/80

(57) ABSTRACT

Disclosed are autonomous work vehicles, systems, and methods for operating an autonomous work vehicle based on plant health data. The method may include driving the autonomous work vehicle through the operating environment, and, while driving the vehicle, receiving multispectral sensor data from a multispectral sensor, determining the plant health value based on the multispectral sensor data, generating a plant health map based on the plant health values and corresponding positioning data, and separating the plant health map into portions based on the plant health values, for example, relative to a health threshold. When the autonomous work vehicle is found within a first portion of the plant health map the vehicle may be operated based on an adjusted set of operating parameters, otherwise the vehicle may be operated based on a default set of operating parameters.

### BACKGROUND

For safe navigation through an environment, autonomous ground vehicles rely on sensory inputs such as cameras, LiDAR, and radar for detection and classification of obstacles and impassable terrain. These sensors provide data representing 3D space surrounding the vehicle.

### SUMMARY

Disclosed are autonomous work vehicles, systems, and methods for operating an autonomous work vehicle based on plant health within the operating environment. This may enable the autonomous work vehicle to perform operational tasks with greater precision and to improve the plant health within the operating environment.

The autonomous work vehicle may comprise a steering control system for autonomously controlling a driving direction of the autonomous work vehicle and a speed control system for autonomously controlling a speed of the autonomous work vehicle. The autonomous work vehicle may include one or more sensors for determining a position of the vehicle. For example, the autonomous work vehicle may comprise a geo positioning sensor that may record or may be used to record geo positioning data.

The autonomous work vehicle may comprise a multispectral sensor that records multispectral sensor data. The multispectral sensor data may be used to determine plant health values of portions within the operating environment. The multispectral sensor may comprise an NDVI camera, an RGB camera, or a multispectral camera. The multispectral sensor may be oriented towards or may extend to a side of the autonomous work vehicle. The autonomous work vehicle may comprise one or more processors communicatively coupled with the multispectral sensor, the steering control system, the speed control system, and the geo positioning sensor. The autonomous work vehicle can comprise one or more computer-readable media having stored thereon instructions that when executed cause the one or more processors to implement a method for driving the autonomous work vehicle based on plant health data.

The method may comprise instructing the steering control system and the speed control system to drive the autonomous work vehicle along a path through the operating environment with a default set of operating parameters. The method may include, while the autonomous work vehicle is driving along the path, receiving and recording multispectral sensor data from the multispectral sensor to the one or more computer-readable media. Thereafter, the autonomous work vehicle may generate a plant health map from the multispectral sensor data and the geo positioning data comprising a virtual representation of at least a portion of the operating environment. Plant health values of different portions of the plant health map may then be determined and stored in the one or more computer-readable media.

The method may then comprise changing at least one of the default set of operating parameters to a first set of operating parameters for a first portion of the plant health map based on data in the plant health map. The autonomous work vehicle may then be operated based on the first set of operating parameters when operating the autonomous work vehicle in the first portion of the plant health map, and may be operated based on the default set of operating parameters when operating the autonomous work vehicle outside the first portion of the plant health map.

The autonomous work vehicle can further comprise an implement and an implement control system for autonomously controlling operation of the implement. The first set of operating parameters may include changing the operation of the implement. For example, the autonomous work vehicle may comprise an autonomous mower, the implement may include a mower reel, and changing at least one of the default set of operating parameters may comprise adjusting a height of the mower reel. In another example, operating the autonomous work vehicle based on the first set of operating parameters may include instructing an implement coupled with or attached to the autonomous vehicle to apply a supplement to a site within the first portion of the health map, wherein the supplement comprises one or more of additional water, fertilizer, pesticide, weed killer, soil, or health-promoting spray.

Changing at least one of the default set of operating parameters may include changing a velocity of the autonomous work vehicle. Changing at least one of the default set of operating parameters to a first set of operating parameters may cause the autonomous vehicle to drive around portions of the operating environment having plant health values below a health threshold.

Generating a plant health map may comprise stitching the multispectral sensor data and the geo positioning data together to create an orthographic map of the operating environment. Generating a plant health map from the multispectral sensor data and the geo positioning data can use a camera projection model. Generating a plant health map from the multispectral sensor data and the geo positioning data may comprise projecting pixels onto a two dimensional map of the operating environment.

Generating a plant health map from the multispectral sensor data and the geo positioning data may include applying a pixel based moving average. Generating a plant health map from the multispectral sensor data and the geo positioning data can comprise applying a Bayesian filter to the multispectral sensor data. Determining plant health from the multispectral sensor data may use deep learning or other image processing techniques.

These illustrative embodiments are mentioned not to limit or define the disclosure, but to provide examples to aid understanding. Additional embodiments are discussed in the Detailed Description, and further description is provided there. Advantages offered by one or more of the various embodiments may be further understood by examining this specification or by practicing one or more embodiments presented.

### BRIEF DESCRIPTION OF THE FIGURES

These and other features, aspects, and advantages of the present disclosure are better understood when the following Detailed Description is read with reference to the accompanying Drawings. In the Drawings, like reference numerals may be utilized to designate corresponding or similar parts in the various Figures, and the various elements depicted are not necessarily drawn to scale, wherein:

FIG. 1 illustrates a block diagram of an example autonomous work vehicle communication system of the present disclosure.

FIG. 2 is a side view of an autonomous yard truck according to some embodiments.

FIG. 3 is a perspective view of an autonomous mower according to some embodiments.

FIG. 4 is a side view of an autonomous tractor according to some embodiments.

FIG. 5 shows an illustrative computational system for performing functionality to facilitate implementation of embodiments described in this document.

FIG. 6 shows a front view of an autonomous tractor, including a multispectral sensor that may be used to determine a plant health of portions of the operating environment.

FIG. 7 illustrates a method for operating an autonomous work vehicle based on the plant health of portions of the operating environment.

FIG. 8 shows a two-dimensional map of an operating environment comprising an agricultural field, with the position of the autonomous tractor and the planned path superimposed on the grid map.

FIG. 9 illustrates a closeup view of the grid map of FIG. 8 showing the autonomous tractor and planned path.

FIG. 10 shows plant health data superimposed on the grid map of FIG. 8.

FIG. 11 illustrates a closeup of the grid map of FIG. 10, including an adjusted path of the autonomous tractor.

FIGS. 12A and 12B illustrate an outline of an autonomous mower similar to the autonomous mower of FIG. 3, showing the height of the mower reel adjusting based on the plant health data.

### DETAILED DESCRIPTION

Autonomous work vehicles have enabled greater precision of performance of operational tasks. Performance may be improved by including more types of data into consideration during autonomous operation of the autonomous work vehicle.

A potential avenue may be to base operational performance of the autonomous work vehicle on plant health within the operating environment. Aerial drones have been used to map the plant health within an operating environment. Sensors, such as normalized difference vegetation index (NDVI) cameras, have been equipped to aerial drones. Data from these sensors may then be correlated with plant density, which may indicate plant health.

Disclosed are autonomous work vehicles and methods for operating autonomous work vehicles based on plant health data. The plant health data may be determined based on multispectral sensor data received from sensors on the autonomous work vehicle. This may remove the necessity of additional hardware in the form of an aerial drone and may enable positioning of the sensors closer to the plant surface, increasing the precision and/or reliability of plant health data. This may also enable the autonomous work vehicle to adjust operation based on current plant health data, for example, to adjust performance of the autonomous work vehicle during the operational task. This disclosure may be applied to agricultural applications, mowing of golf courses or sod farms, or other contexts.

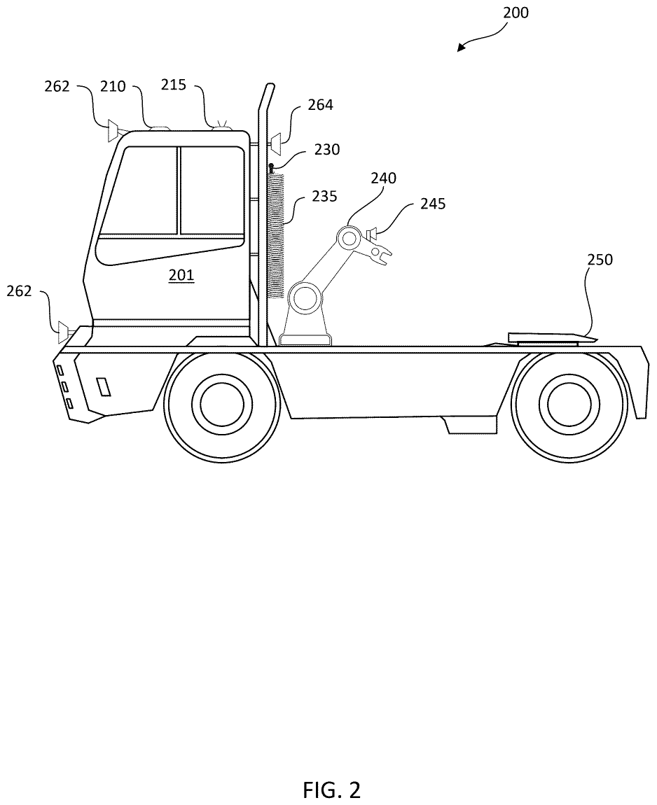

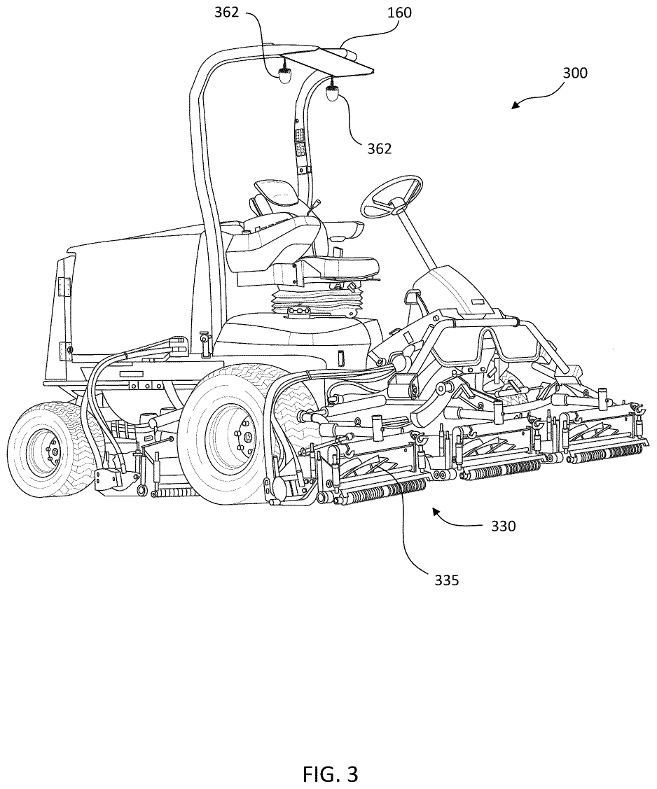

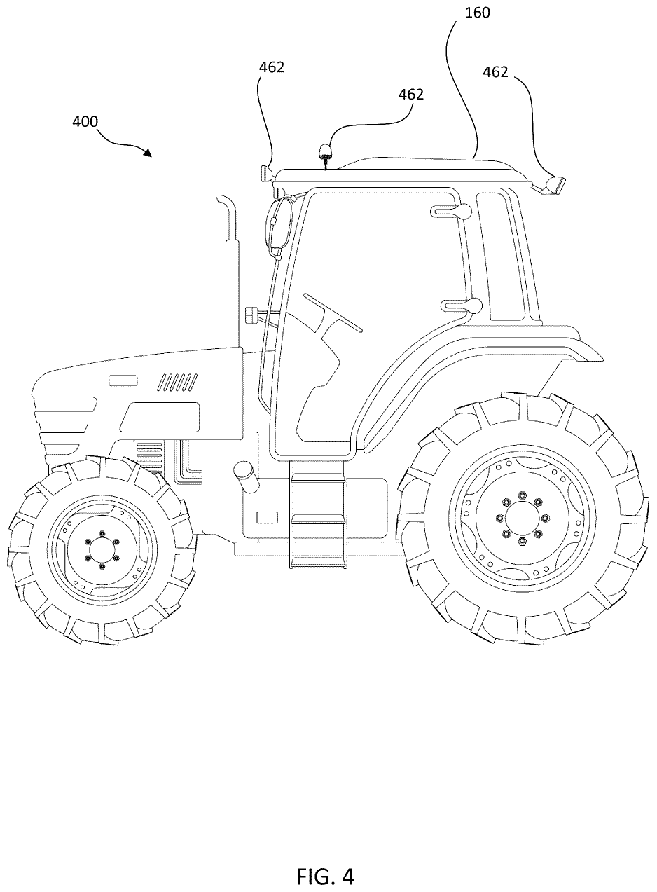

As used herein, the term “autonomous work vehicle” may refer to a vehicle configured to perform a task within an operating environment. The autonomous work vehicle may refer to a vehicle that may be driven by an autonomous system with little or no human input. The autonomous work vehicle may refer to a driving unit (e.g., a tractor) attached to an implement, or may refer to the driving unit as distinct from the implement. Examples of autonomous work vehicles are shown in FIG. 2, FIG. 3, and FIG. 4. The terms “autonomous work vehicle” and “autonomous tractor” may be used interchangeably throughout the document.

As used herein, the term “implement” may refer to a device connected (detachably or permanently) to an autonomous work vehicle and which is configured to interface and/or interact with objects (beyond the vehicle to which it is connected) within the operating environment. The implement may include farming implements, construction implements, or other vehicle attachments. The implement may include a vehicle connection point for detachably and/or pivotably connecting the implement to a vehicle (e.g., an autonomous work vehicle). The implement may be configured, for example, to move objects within the operating environment, to cut or partition objects, or to bind objects together. The implement may be configured to interface with a ground surface of the operating environment, vegetation, crops, sod, grass, etc.

As used herein, the term “autonomous vehicle system,” “autonomous work vehicle system,” or related terms may refer to the combination of the autonomous work vehicle hardware (including sensors), an attached implement (including sensors), and the software that enables the vehicle to perform an operational task autonomously. The term may refer to the autonomous work vehicle and an attached implement, and/or may include the software and hardware for executing the methods described below and/or the computer-readable media storing such methods. In some embodiments, the term may additionally include the base station that communicates with the autonomous work vehicle. In some embodiments, the term may additionally include a kit that can be installed on a vehicle that may allow the vehicle to operate autonomously.

As used herein, the term “operator” may refer to a manual driver of the work vehicle, who may be present within the cab of the vehicle or may refer to a remote operator that drives the work vehicle from a distance. The operator may refer to a remote operator that may receive sensor data from the work vehicle to drive, manage, and/or troubleshoot the work vehicle from a distance. For example, if the autonomous work vehicle pauses or stops (e.g., due to encountering an unknown situation or an operational or sensor failure), a remote operator may receive sensor data (e.g., an image of an unexpected obstacle) of the operating environment, and the remote operator may then upon examination of the sensor data configure the autonomous work vehicle to resume operation. In some embodiments, the operator may refer to a technician that services the work vehicle for maintenance and repair, and/or who may troubleshoot the work vehicle, especially during operational pauses of the work vehicle (e.g., autonomous work vehicle).

As used herein, the term “operating parameter” may refer to a setting that may be directly adjusted (e.g., by a control system of the autonomous work vehicle and/or implement) during performance of an operational task of the autonomous work vehicle system. The setting may be individually adjusted or may be adjusted in combination with one or more settings. For example, the operational parameter(s) may include a velocity, a wheel speed, engine torque, fuel burn rate, traction, wheel slip, engine rpm, steering effectiveness, and/or a heading or path of the autonomous work vehicle, or may include a height, penetration depth, extension, speed, position, hydraulic fluid volume, hydraulic fluid flow rate, and/or power input of an attached implement.

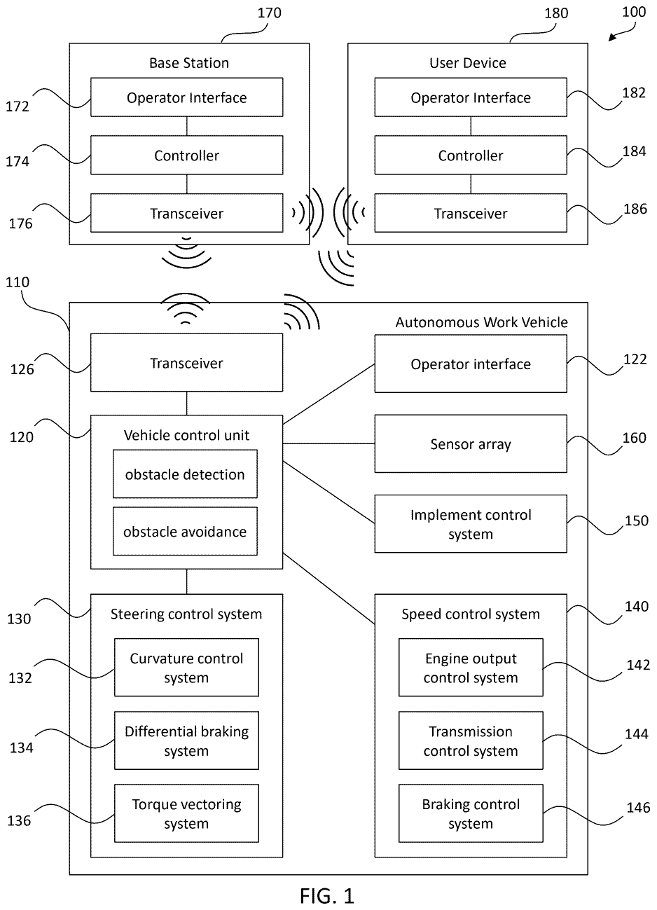

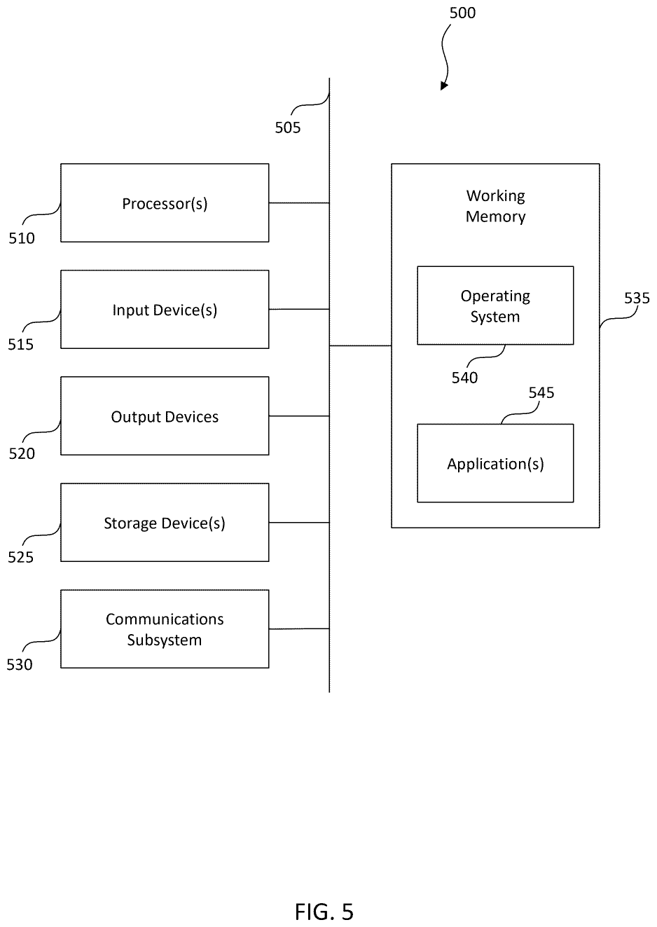

FIG. 1 is a block diagram of a communication and control system 100 that may be utilized in conjunction with the systems and methods of the disclosure. The communication and control system 100 may include a vehicle control unit 120 which may be mounted on an autonomous work vehicle 110. The autonomous work vehicle 110, for example, may include a yard truck, loader, wheel loader, track loader, dump truck, digger, backhoe, forklift, mower (e.g., lawn, field, or brush mower), or other work vehicle. The communication and control system 100, for example, may include any or all components of computational system 500 shown in FIG. 5, described below.

For example, the autonomous work vehicle 110 may include a steering control system 130 that may control a direction of movement of the autonomous work vehicle 110. The steering control system 130, for example, may include any or all components of computational system 500 shown in FIG. 5.

The autonomous work vehicle 110, for example, may include a speed control system 140 that controls the speed, acceleration, and deceleration of the autonomous work vehicle 110. The speed control system 140, for example, may control the speed of the autonomous work vehicle 110 based on map data, control algorithms, obstacle detection, start and/or stop points, input from the operator (e.g., a remote operator), etc. The speed control system 140, for example, may include any or all components of computational system 500 shown in FIG. 5.

The autonomous work vehicle 110, for example, may include an implement control system 150 that may control operation of an implement towed by the autonomous work vehicle 110, integrated within the autonomous work vehicle 110, or coupled to the autonomous work vehicle 110. The implement control system 150, for example, may include any type of implement such as, for example, a bucket, a shovel, a blade, a thumb, a dump bed, a plow, an auger, a trencher, a scraper, a broom, a hammer, a grapple, forks, boom, spears, a cutter, a wrist, a tiller, a rake, etc. The implement control system 150, for example, may include any or all components of computational system 500 shown in FIG. 5.

The vehicle control unit 120 may be communicatively coupled with the steering control system 130, the speed control system 140, and/or the implement control system 150. The vehicle control unit 120, for example, may include any or all of the components shown in FIG. 5. The vehicle control unit 120, for example, may be integrated into a single controller or may include a plurality of distinct components or controllers. The vehicle control unit 120 may also be coupled with one or more sensors from the sensor array 160 and receive sensor data from the sensor array 160.

The vehicle control unit 120, for example, may be used to control various aspects of the vehicle 110 such as, for example, sending instructions to the steering control system 130, implement control system 150, speed control system 140, etc. The vehicle control unit 120, for example, may include a vehicle artificial intelligence (VAI) that may include one or more processors that execute one or more algorithms, including method 700 disclosed below.

The vehicle control unit 120, for example, may receive signals relative to many parameters of interest including, but not limited to: vehicle position, vehicle speed, vehicle heading, desired path location, off-path normal error, desired off-path normal error, heading error, vehicle state vector information, curvature state vector information, turning radius limits, steering angle, steering angle limits, steering rate limits, curvature, curvature rate, rate of curvature limits, roll, pitch, rotational rates, acceleration, and the like, or any combination thereof. These signals, for example, may come from the sensor array 160 or from a base station 170 (described below).

The vehicle control unit 120, for example, may be an electronic controller with electrical circuitry configured to process data from the various components of the autonomous work vehicle 110. The vehicle control unit 120 may include a processor, such as the processor 510, and a working memory 535. The vehicle control unit 120 may also include one or more storage devices, storage media, and/or other suitable components of computational system 500. The processor may be used to execute software, such as software for calculating drivable path plans. Moreover, the processor may include multiple microprocessors, one or more “general-purpose” microprocessors, one or more special-purpose microprocessors, and/or one or more application specific integrated circuits (ASICS), or any combination thereof. For example, the processor may include one or more reduced instruction set (RISC) processors. The vehicle control unit 120, for example, may include any or all the components shown in FIG. 5.

The vehicle control unit 120, for example, may include a volatile memory, such as random access memory (RAM), and/or a nonvolatile memory, such as ROM (e.g., working memory 535, storage device 525, and/or other computer-readable media). The memory may store a variety of information and may be used for various purposes. For example, the memory may store processor-executable instructions (e.g., firmware or software) for the vehicle control unit 120 to execute, such as instructions for calculating a drivable path plan, and/or controlling the autonomous work vehicle 110 (e.g., for implementing method 700 below). The memory may include flash memory, one or more hard drives, or any other suitable optical, magnetic, or solid-state storage medium, or a combination thereof. The memory may store data such as field maps, maps of desired paths, vehicle characteristics, software or firmware instructions, and/or any other suitable data.

The steering control system 130, for example, may include a curvature rate control system 132, a differential braking system 134, a steering mechanism, and a torque vectoring system 136 that may be used to steer the autonomous work vehicle 110. The curvature rate control system 132, for example, may control a direction of an autonomous work vehicle 110 by controlling a steering control system of the autonomous work vehicle 110 with a curvature rate, such as an Ackerman style autonomous work vehicle, 110 or articulating vehicle. The curvature rate control system 132, for example, may automatically rotate one or more wheels or tracks of the autonomous work vehicle 110 via hydraulic or electric actuators to steer the autonomous work vehicle 110. By way of example, the curvature rate control system 132 may rotate front wheels/tracks, rear wheels/tracks, and/or intermediate wheels/tracks of the autonomous work vehicle 110 or articulate the frame of the vehicle, either individually or in groups. The differential braking system 134 may independently vary the braking force on each lateral side of the autonomous work vehicle 110 to direct the autonomous work vehicle 110. Similarly, the torque vectoring system 136 may differentially apply torque from the engine to the wheels and/or tracks on each lateral side of the autonomous work vehicle 110. While the illustrated steering control system 130 includes the curvature rate control system 132, the differential braking system 134, and the torque vectoring system 136, the steering control system 130 may include one or more of these systems. Further examples may include a steering control system 130 having other and/or additional systems to facilitate turning the autonomous work vehicle 110 such as an articulated steering control system, a differential drive system, and the like.

The speed control system 140, for example, may include an engine output control system 142, a transmission control system 144, and a braking control system 146. The engine output control system 142 may vary the output of the engine to control the speed of the autonomous work vehicle 110. For example, the engine output control system 142 may vary a throttle setting of the engine, a fuel/air mixture of the engine, a timing of the engine, and/or other suitable engine parameters to control engine output. In addition, the transmission control system 144 may adjust gear selection within a transmission to control the speed of the autonomous work vehicle 110. Furthermore, the braking control system 146 may adjust the braking force to control the speed of the autonomous work vehicle 110. While the illustrated speed control system 140 includes the engine output control system 142, the transmission control system 144, and the braking control system 146, the speed control system 140 may include one or two of these systems. The speed control system 140, for example, may also include other systems and/or additional systems that may be used to control the speed of the autonomous work vehicle 110.

The implement control system 150, for example, may control various parameters of the implement towed by and/or integrated within the autonomous work vehicle 110. For example, the implement control system 150 may instruct an implement controller via a communication link, such as a CAN bus, ISOBUS, Ethernet, wireless communications, and/or Broad R Reach type Automotive Ethernet, etc.

The implement control system 150, for example, may instruct an implement controller to adjust a penetration depth of at least one ground engaging tool of an agricultural implement, which may reduce the draft load on the autonomous work vehicle 110.

The implement control system 150, as another example, may instruct the implement controller to transition an agricultural implement between a working position and a transport portion, to adjust a flow rate of product from the agricultural implement, to adjust a position of a header of the agricultural implement (e.g., a harvester, etc.), among other operations, etc. The implement control system 150, as another example, may instruct the implement controller to adjust a shovel height, a shovel angle, a shovel position, etc.

The communication and control system 100, for example, may include a sensor array 160. The sensor array 160, for example, may facilitate determination of condition(s) of the autonomous work vehicle 110 and/or the work area. For example, the sensor array 160 may include one or more sensors (e.g., infrared sensors, ultrasonic sensors, magnetic sensors, tachometer, radar sensors, LiDAR sensors, terahertz sensors, sonar sensors, wheel encoders, cameras, etc.) that monitor a rotation rate of a respective wheel and/or track a ground speed of the autonomous work vehicle 110. The sensors may also monitor operating levels (e.g., temperature, fuel level, etc.) of the autonomous work vehicle 110. Furthermore, the sensors may monitor conditions in and around the work area, such as temperature, weather, wind speed, compass, humidity, and other conditions. The sensors of the sensor array 160, for example, may detect physical objects in the work area, such as a parking stall, a material stall, accessories, other vehicles, obstacles, environmental features, or other object(s) that may be in the area surrounding the autonomous work vehicle 110.

The sensor array 160, for example, may include a velocity sensor which may include one or more of an inertial measurement unit, a compass, a GPS sensor, a wheel encoder, a tachometer, a camera, a radar, etc. The sensor array 160, for example, may also include a steering angle sensor. The velocity sensor, for example, may produce velocity data. Velocity data may include information regarding speed and/or bearing. Velocity data, for example, may additionally, or alternatively, include information regarding the steering angular rate.

The autonomous work vehicle 110 may include an operator interface 122 for controlling the vehicle. The operator interface 122, for example, may be communicatively coupled to the vehicle control unit 120 and configured to present data from the autonomous work vehicle 110 via a display. Display data may include data associated with operation of the autonomous work vehicle 110, data associated with operation of an implement, a position of the autonomous work vehicle 110, a speed of the autonomous work vehicle 110, a desired path, a drivable path plan, a target position, and/or a current position, etc. The operator interface 122 may enable an operator to control certain functions of the autonomous work vehicle 110 such as starting and stopping the autonomous work vehicle 110, inputting a desired path, etc. The operator interface 122, for example, may enable the operator to input parameters that cause the vehicle control unit 120 to adjust the drivable path plan. For example, the operator may provide an input requesting that the desired path be acquired as quickly as possible, that an off-path normal error be minimized, that a speed of the autonomous work vehicle 110 remain within certain limits, and/or that a lateral acceleration experienced by the autonomous work vehicle 110 remain within certain limits, etc. In addition, the operator interface 122 (e.g., via the display, or via an audio system (not shown), etc.) may alert an operator if the desired path cannot be achieved, for example.

The communication and control system 100, for example, may include a base station 170 having a base station controller 174 located remotely from the autonomous work vehicle 110. For example, the control functions of the vehicle control unit 120 may be distributed between the vehicle control unit 120 of the autonomous work vehicle 110 and the base station controller 174. The base station controller 174, for example, may perform a substantial portion of the control functions of the vehicle control unit 120. For example, a first transceiver 126 positioned on the autonomous work vehicle 110 may output signals indicative of vehicle characteristics (e.g., position, speed, heading, curvature rate, curvature rate limits, maximum turning rate, minimum turning radius, steering angle, roll, pitch, rotational rates, acceleration, etc.) to a second transceiver 176 at the base station 170. The base station controller 174, for example, may calculate drivable path plans and/or output control signals to control the curvature control system 132, the speed control system 140, and/or the implement control system 150 to direct the autonomous work vehicle 110 toward the desired path, for example. The base station controller 174 may include a processor and memory device having similar features and/or capabilities as the processor and the memory device discussed previously. Likewise, the base station 170 may include an operator interface 172 having a display, which may have similar features and/or capabilities as the operator interface 122 and the display discussed previously.

In some embodiments, one or both of the base station 170 and/or the autonomous work vehicle 110 may be in communication with a user device 180. A user device 180 may include a phone, tablet, laptop, or computer. The user device 180 may similarly include an operator interface 182 which may include similar features and capabilities as operator interfaces 122, 172 described above. Additionally, or alternatively, the user device 180 may comprise a controller 184 that may include the same or similar features, components, and/or characteristics as the controller 174 of the base station 170. For example, the user device controller 174 may calculate drivable path plans, output control signals to control the curvature control system 132, the speed control system 140, and/or the implement control system 150 to direct the autonomous work vehicle 110. The user device 180, for example, can include an application that allows the user (e.g., a remote operator) to communicate commands to the autonomous work vehicle 110 (e.g., via a transceiver 186) and/or receive information about the autonomous work vehicle 110. Alternatively, or additionally, the user device 180, for example, can include an application that allows the operator to observe the autonomous work vehicle 110 move through a map of the work area where the autonomous work vehicle operates.

The user device 180, for example, may include an application that can receive an indication associated with the remote operator or which can receive other user or operator inputs. The user device 180, for example, may include an application that can display any of the information disclosed in this document.

FIG. 2 is a side view of an autonomous yard truck 200 according to some embodiments. The autonomous yard truck 200 includes a cab 201 that may be used to drive the autonomous yard truck 200 manually. The autonomous yard truck 200 may include one or more of the components shown in FIG. 1. The autonomous yard truck 200 may also include a brake system, an engine, a transmission, steering, sensor array, etc. such as, for example, as shown in FIG. 1.

In some embodiments, the autonomous yard truck 200 may include a sensor array that includes sensors 262 (e.g., sensor array 160) disposed at various locations on the autonomous yard truck 200 such as, for example, on the cab 201, bumper, housing, frame, etc. The sensors 262 may include infrared sensors, ultrasonic sensors, magnetic sensors, radar sensors, LiDAR sensors, terahertz sensors, sonar sensors, cameras, etc. The sensor array of the autonomous yard truck 200 may also include one or more backup sensors 264 such as, for example, infrared sensors, ultrasonic sensors, magnetic sensors, radar sensors, LiDAR sensors, terahertz sensors, sonar sensors, cameras, etc.

In some embodiments, the autonomous yard truck 200 may include a spatial locating device (or GPS) antenna 210. In some embodiments, the autonomous yard truck 200 may include a transceiver antenna 215.

In some embodiments, the autonomous yard truck 200 may include one or more hoses 235 that can connect with a trailer such as, for example, two or three hoses. Each hose may have a hose connector 230 that can connect with a trailer hose connector. For example, the one or more hoses 235 of the autonomous yard truck 200 may include a service brake hose, an emergency brake hose, and/or a refrigerant hose.

In some embodiments, the autonomous yard truck 200 may include a robotic arm 240 disposed on the back bed of the autonomous yard truck 200. The robotic arm 240 may include any type of robotic arm. The robotic arm 240, for example, may exert high torque or high pressure sufficient to connect the hose connector 230 with the trailer hose connector. The hose connector 230 and/or the trailer hose connector may comprise a glad-hand connector. In some embodiments, when the autonomous yard truck 200 is not coupled with a trailer, the hose connector 230 may be positioned in a storage rack at some point on the autonomous yard truck 200 such as, for example, on the rear of the cab 201.

In some embodiments, the robotic arm 240 may include one or more arm sensors 245 such as, for example, infrared sensors, ultrasonic sensors, magnetic sensors, radar sensors, LiDAR sensors, terahertz sensors, sonar sensors, cameras, etc. The arm sensor 245, for example, may produce data that can be used to identify the location of a hose connector 230 and/or a trailer hose connector. The arm sensor 245, for example, may produce data that can show that a hose connector 230 and/or a trailer hose connector are sufficiently coupled.

In some embodiments, the autonomous yard truck 200 may include a fifth-wheel coupling 250. The fifth-wheel coupling 250, for example, may be raised or lowered with a fifth-wheel coupling boom. FIG. 2 shows the fifth-wheel coupling 250 in a lowered position. The fifth-wheel coupling 250 may couple with a kingpin of a trailer.

When the fifth-wheel coupling 250 is coupled with a kingpin and the fifth-wheel coupling 250 is in the raised position, the legs of the trailer may lift off the ground (e.g., automatically). This may allow the autonomous yard truck 200 to pull the trailer without individually raising the trailer legs.

In some embodiments, the robotic arm 240 and/or the arm sensor 245 may be coupled with a thermal management system. A thermal management system may, for example, be coupled with a thermal management system associated with the autonomous yard truck 200 such as, for example, coupled with the cab heating/cooling system and/or the engine heating/cooling system. A thermal management system may, for example, be an independent system that heats and/or cools the robotic arm 240 and/or the arm sensor 245. A thermal management system may, for example, keep the temperature of the robotic arm 240 and/or the arm sensor 245 between about 32 °F and about 100 °F.

In some embodiments, the autonomous yard truck 200 may include a deployable shade coupled with the back of the cab 201. The deployable shade, for example, may be used to screen the sun and/or other lighting from the arm sensor 245 and/or the one or more backup sensors 264. The deployable shade, for example, may include an umbrella configuration or an awning configuration. The deployable shade, for example, may be coupled with the roof or an upper portion of the cab.

FIG. 3 is a sideview of an example autonomous mower 300, which may include all or some of the components of autonomous work vehicle 110. The autonomous work vehicle in this document may include the autonomous mower 300. Any type of mower or blades may be used, such as a disc mower. The autonomous mower 300, for example, may include a sensor array 160 (or multiple sensor arrays 160), including sensors 362. The sensor array 160 may include, for example, one or more LiDAR, radar, and/or video cameras. The video cameras, for example, may include 260 degree cameras, a front facing camera, and/or a back facing camera. The autonomous mower 300 may include a mower reel 330, including a reel blade 335 for cutting the surface of the operating environment.

FIG. 4 is a sideview of an example autonomous tractor 400, which may include all or some of the components of autonomous work vehicle 110. The autonomous work vehicle in this document may include the autonomous tractor 400. In this example, the autonomous tractor 400 may include standard tractor equipment and/or components. The autonomous tractor 400 may include or be coupled with any kind of implement such as, for example, a plow, disc plow, reel mower, dumper, lift, bucket, shovel, blade, and/or cutter, etc. The autonomous tractor 400, for example, may include a sensor array 160 (or multiple sensor arrays 160), including sensor(s) 462. The sensor array 160 may include, for example, one or more LiDAR, radar, and/or video cameras. The video cameras, for example, may include 260 degree cameras, a front facing camera, and/or a back facing camera.

The computational system 500, shown in FIG. 5, can be used to perform any of the embodiments of the invention. For example, computational system 500 can be used to execute method 700. As another example, computational system 500 can be used to perform any calculation, identification, and/or determination described here. Computational system 500 includes hardware elements that can be electrically coupled via a bus 505 (or may otherwise be in communication, as appropriate). The hardware elements can include one or more processors 510, including without limitation one or more general-purpose processors and/or one or more special-purpose processors (such as digital signal processing chips, graphics acceleration chips, and/or the like); one or more input devices 515, which can include without limitation a mouse, a keyboard, and/or the like; and one or more output devices 520, which can include without limitation a display device, a printer, and/or the like.

The computational system 500 may further include (and/or be in communication with) one or more storage devices 525, which can include, without limitation, local and/or network accessible storage and/or can include, without limitation, a disk drive, a drive array, an optical storage device, a solid-state storage device, such as a random access memory (“RAM”) and/or a read-only memory (“ROM”), which can be programmable, flash-updateable, and/or the like. The computational system 500 might also include a communications subsystem 530, which can include without limitation a modem, a network card (wireless or wired), an infrared communication device, a wireless communication device and/or chipset (such as a Bluetooth device, an 802.6 device, a Wi-Fi device, a WiMax device, cellular communication facilities, etc.), and/or the like. The communications subsystem 530 may permit data to be exchanged with a network (such as the network described below, to name one example), and/or any other devices described herein. In many embodiments, the computational system 500 will further include a working memory 535, which can include a RAM or ROM device, as described above.

The computational system 500 also can include software elements, shown as being currently located within the working memory 535, including an operating system 540 and/or other code, such as one or more application programs 545, which may include computer programs of the invention, and/or may be designed to implement methods of the invention and/or configure systems of the invention, as described herein. For example, one or more procedures described with respect to the method(s) discussed above might be implemented as code and/or instructions executable by a computer (and/or a processor within a computer). A set of these instructions and/or codes might be stored on a computer-readable storage medium, such as the storage device(s) 525 described above.

In some cases, the storage medium might be incorporated within the computational system 500 or in communication with the computational system 500. In other embodiments, the storage medium might be separate from a computational system 500 (e.g., a removable medium, such as a compact disc, etc.), and/or provided in an installation package, such that the storage medium can be used to program a general-purpose computer with the instructions/code stored thereon. These instructions might take the form of executable code, which is executable by the computational system 500 and/or might take the form of source and/or installable code, which, upon compilation and/or installation on the computational system 500 (e.g., using any of a variety of generally available compilers, installation programs, compression/decompression utilities, etc.) then takes the form of executable code.

The computational system 500 may be configured to operate an autonomous work vehicle platform. The term “autonomous work vehicle”, and related terms (e.g., “autonomous work vehicle platform”), as used herein may include manned vehicles, remote control vehicles, and/or manual vehicles, etc. The autonomous work vehicle platform may comprise a steering mechanism in communication with the processor, where the processor communicates steering commands to the steering mechanism based on plant health data. The autonomous work vehicle platform may comprise a braking mechanism in communication with the processor, where the processor communicates braking commands to the braking mechanism based on plant health data.

The autonomous work vehicle 110 may be operated based on the plant health of vegetation within the operating environment. The plant health data may be collected by the autonomous work vehicle 110 during the performance an operational task. An operational setting of the autonomous work vehicle 110 may then be altered (e.g., when compared to a default or predetermined setting) to adjust the performance of the operational task. Performance of the autonomous work vehicle 110 may be adjusted based on the plant health data. For example, the performance of the operational task may be based on the plant health of the site or portion of the operating environment in which the autonomous work vehicle 110 is operating.

A velocity, position, and/or orientation of the autonomous work vehicle 110 may be adjusted based on the plant health data. In some embodiments, the position, orientation, movement, or other settings of an implement may be adjusted based on the plant health data. The path followed by the autonomous work vehicle 110 may be adjusted based on the plant health data. The adjusted performance of the autonomous work vehicle 110 may be calculated to improve the plant health of the vegetation of portions of the operating environment, such as portions of the operating environment having plant health values lying below a health threshold. Described below are methods and systems for operating the autonomous work vehicle 110 based on the plant health data.

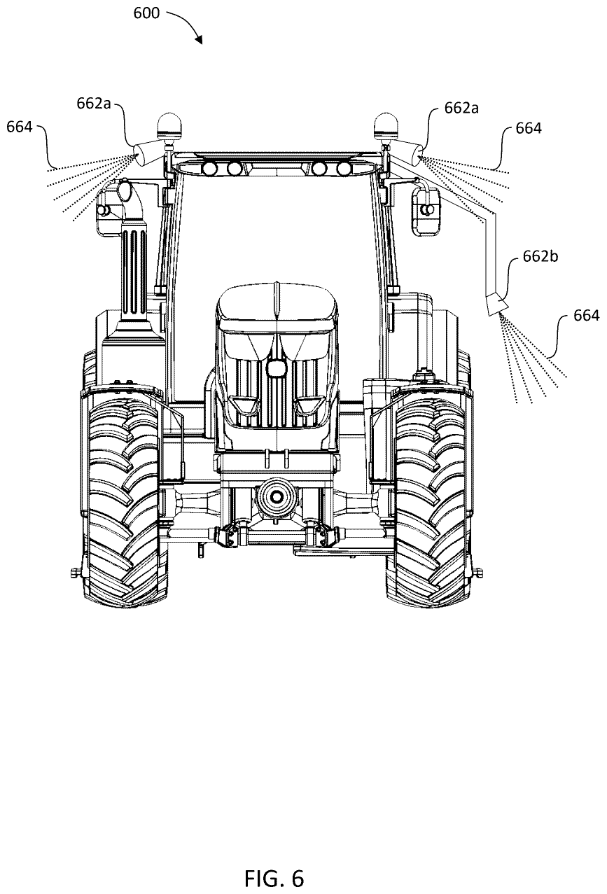

FIG. 6 illustrates a front view of an autonomous work vehicle 110 comprising an autonomous tractor 600, which may be similar to and/or comprise all the characteristics of autonomous tractor 400 described above, which may form part of the autonomous vehicle system, and which may be used to operate in, determine plant health values of vegetation within, and adjust performance based on the plant health values of an operating environment comprising an agricultural field or environment. The autonomous tractor 600 may comprise a positioning sensor for determining a position of the autonomous work vehicle 110 within the operating environment. For example, the autonomous tractor 600 may comprise a geo positioning sensor, which may generate positioning data (i.e., geo positioning data) of the autonomous tractor 600. The autonomous work vehicle 110 and/or the positioning sensor (e.g., geo positioning sensor) itself may record the positioning data (e.g., geo positioning data). In some embodiments, odometry data may be used to determine the position of the autonomous work vehicle 110 within the operating environment.

The autonomous tractor 600 may include one or more multispectral sensors 662a, 662b that record multispectral sensor data. The multispectral sensors 662a, 662b may form part of the sensor array 160. The one or more multispectral sensors 662a, 662b may comprise a camera that can produce data that can be used to determine characteristics of plants associated with plant health. For example, the multispectral sensor may comprise an NDVI camera, an RGB camera, a multispectral camera, a hyperspectral camera, an infra-red camera, UV camera, or other camera.

The multispectral sensors 662a, 662b may be positioned on the autonomous tractor 600 at a location that enables the sensor field of view (FOV) 664 includes portions of the operating environment lying close to and/or surrounding the autonomous tractor 600. For example, the multispectral sensors 662a, 662b may be positioned or extend from a top end of the autonomous work vehicle 110, such as the upper surface of the cab of the autonomous work vehicle 110. The sensor FOV 664 of the multispectral sensors 662a, 662b may include portions of the operating environment lying in front of, behind, or to the side of the autonomous tractor 600. In some embodiments, the sensor FOV 664 may extend in 360 degrees around the autonomous work vehicle 110, or may extend 270 degrees, or 180 degrees, or 120 degrees, or 90 degrees from the autonomous work vehicle 110, or may extend within a range having any two of the foregoing as endpoints.

The multispectral sensor 662a, 662b may be configured to measure data that can be interpreted to indicate relative plant health of portions of the operating environment lying to a side of the autonomous work vehicle. For example, the one or more multispectral sensors may be oriented towards or extend to a side of the autonomous work vehicle 110 to better determine the plant health of portions of the operating environment lying towards the side of the autonomous work vehicle 110. This may enable the improved detection of plant health by the multispectral sensors 662a, 662b. For example, implements attached to the front or rear ends of the autonomous tractor 600 may occlude the operating environment from the sensor FOV 664. Additionally, dust or other debris may be generated by the autonomous work vehicle 110 during the operation which may further occlude portions of the operating environment lying to the rear of the autonomous work vehicle 110.

In some embodiments, the autonomous tractor 600 may include multispectral sensors 662a that may be oriented towards the sides (i.e., the right side and/or the left side) of the autonomous work vehicle 110, such that the FOV 664 of the multispectral sensors 662a extends towards a side of the autonomous work vehicle 110. In some embodiments, the autonomous tractor 600 may additionally, or alternatively, include multispectral sensor(s) 662b that extend from the side (and/or top end) of the autonomous tractor 600. This may enable the multispectral sensor(s) 662b to be positioned closer to the vegetation of the operating environment and may enable a more accurate determination of the plant health. The autonomous tractor 600 may comprise one multispectral sensor 662a, 662b, or may comprise multiple multispectral sensors 662a, 662b, such two, three, four, five, six, seven, eight, nine, ten, or more than ten multispectral sensors 662a, 662b. The autonomous tractor 600 may comprise one or more than one multispectral sensors 662a, 662b oriented towards and/or extending from each side of the autonomous tractor 600. In some embodiments, the multispectral sensor data received from the multispectral sensor 662a, 662b may be used to generate a path through the operating environment to navigate the autonomous work vehicle 110.

The one or more processors 510 of the autonomous work vehicle 110 may be communicatively coupled with the (e.g., one or more) multispectral sensor(s) 662a, 662b, the steering control system, the speed control system, and the geo positioning sensor to operate the autonomous work vehicle 110. In some embodiments, the autonomous work vehicle 110 may further comprise an implement and an implement control system for autonomously controlling the operation of the implement (and, e.g., communicatively coupled with the one or more processors 510). The implement may then be adjusted based on the plant health data.

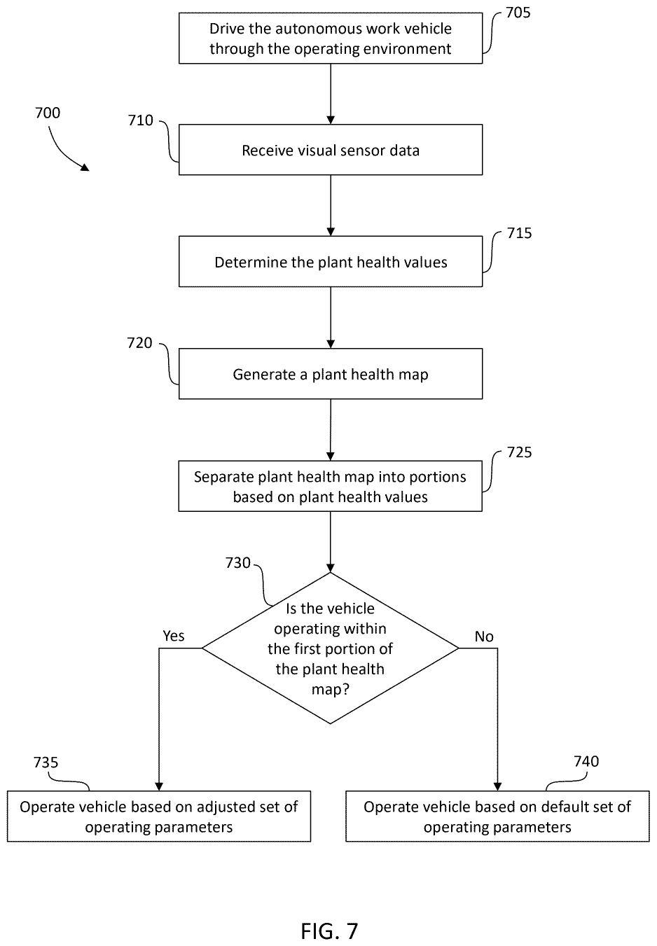

FIG. 7 shows a flowchart of a method 700 for operating an autonomous work vehicle 110 based on the plant health data. The method may begin at block 705 where the autonomous work vehicle 110 is driven along a path through the operating environment (e.g., based on instructions to the steering control system and the speed control system). The autonomous work vehicle 110 may be driven with a default set of operating parameters. The default set of operating parameters may include parameters such as, for example, a speed, an implement height, an implement speed, an implement setting, an implement spray setting (e.g., on, off, volume, direction, spray angle, etc.)

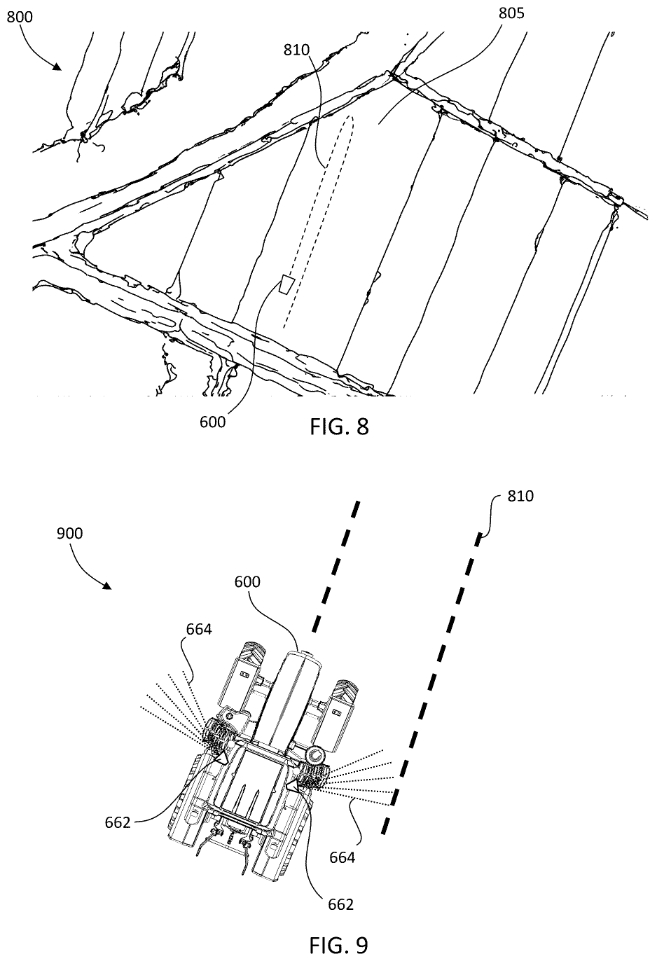

FIG. 8 illustrates a two-dimensional map 800 of the operating environment comprising an agricultural field 805 in which the autonomous tractor 600 may operate. The two-dimensional map 800 may have been previously generated and may be used by the autonomous vehicle system (e.g., within a virtual representation) to navigate the autonomous work vehicle 110 through the operating environment. The agricultural field 805 may comprise vegetation of interest to the operational task, such as a crop. The two-dimensional map 800 further illustrates the position of the autonomous tractor 600 within the agricultural field 805 and a portion of a path 810 which the autonomous tractor 600 may follow during performance of the operational task, superimposed upon the map 800. The operational task may be performed to prepare for or promote the growth of vegetation within the operating environment. For example, the autonomous tractor 600 may perform plowing, tilling, fertilizing, watering, spraying, or harvesting within the agricultural field 805.

FIG. 9 shows a close up view 900 of FIG. 8, illustrating the autonomous tractor 600 as it follows the path 810. Method 700 may proceed to block 710, in which as the autonomous work vehicle 110 follows the path 810 (e.g., the first path), the autonomous work vehicle 110 may receive (and/or record to, for example, the one or more computer-readable media) multispectral sensor data from the one or more multispectral sensors 662. In this manner, the multispectral sensor data may be collected during performance of the operational task. This may remove the necessity of an additional vehicle (such as an aerial drone) for measuring plant health within the operating environment, which may reduce the hardware and/or software requirements to perform the task and reduce operational costs.

At block 715 plant health values of different portions of the operating environment are generated based on the multispectral sensor data. For example, the multispectral sensor data may comprise data from an NDVI camera, and the plant health values may be correlated with the signal (e.g., magnitude and/or spectrum.) of the readings from the NDVI camera. The term “plant health value” as used herein may represent a signal data from the multispectral sensor in raw, filtered, and/or processed form that can be used to indicate the relative health of vegetation. In another example, the multispectral sensor data may comprise data received from an infra-red camera, and the plant health value may be inversely proportional to the value of the readings from the infra-red camera.

In some embodiments, the plant health value can be determined by comparing the multispectral sensor data to a collection of images having pre-calculated plant health values, and the determined plant health value may correlate to the pre-calculated plant health values to the degree that that the multispectral sensor data matches and/or reflects an image from the collection of images. In some embodiments, the plant health values may be determined based on output from a machine learning, deep learning, or other image processing techniques.

The plant health value may be associated with a particular site or region of the operating environment. The plant health value may be stamped and/or correlated with positioning data (e.g., geo positioning data or odometry data). The plant health value may be associated with a particular location or site of the operating environment. For example, a virtual representation of the operating environment may be organized into a grip map comprising a plurality of grid cells, and the plant health value may correspond to the plant health within a particular grid cell. In some embodiments, the plant health value may be generated from an average of data points within the multispectral sensor signal data, such that the plant health value represents an average plant health of plants or vegetation within the grid cell or region of the operating environment. The plant health values may then be stored in the one or more computer-readable media.

The method 700 may then proceed to block 720 in which a plant health map is generated. Generation of the plant health map may be based on the plant health values and the positioning data (e.g., geo positioning data). The plant health map may comprise a virtual representation of at least a portion of the operating environment. Generating the plant health map may comprise stitching the plant health values and the positioning data (e.g., geo positioning data) together to create an orthographic map of the operating environment. The plant health values may be correlated with positioning data and then overlayed on to the two-dimensional map 800 to generate the plant health map.

In some embodiments, the plant health map may resemble a heat map and the autonomous work vehicle 110 may be operated based on the plant health values projected within the plant health map and/or the position of the autonomous work vehicle 110 relative to portions of the operating environment associated with the plant health values within the plant health map.

Generating the plant health map from the plant health values and the positioning data (e.g., geo positioning data) may further comprise projecting pixels onto the two-dimensional map 800 of the operating environment. Generating the plant health map 1000 may comprise applying a pixel based moving average. In some embodiments, generating the plant health map 1000 from the plant health values and the geo positioning data may use a camera projection model and/or may use an inverse camera projection model. The inverse camera projection model made be used assuming a flat plane, or may use extrinsically-and intrinsically-calibrated 3D sensors to geo-position the pixel data. A Bayesian filter may be applied to the multispectral sensor data and/or to the plant health values to generate the plant health map 1000. The method 700 may continue to block 725, wherein the plant health map is defined and/or separated into portions, for example, based on a health threshold.

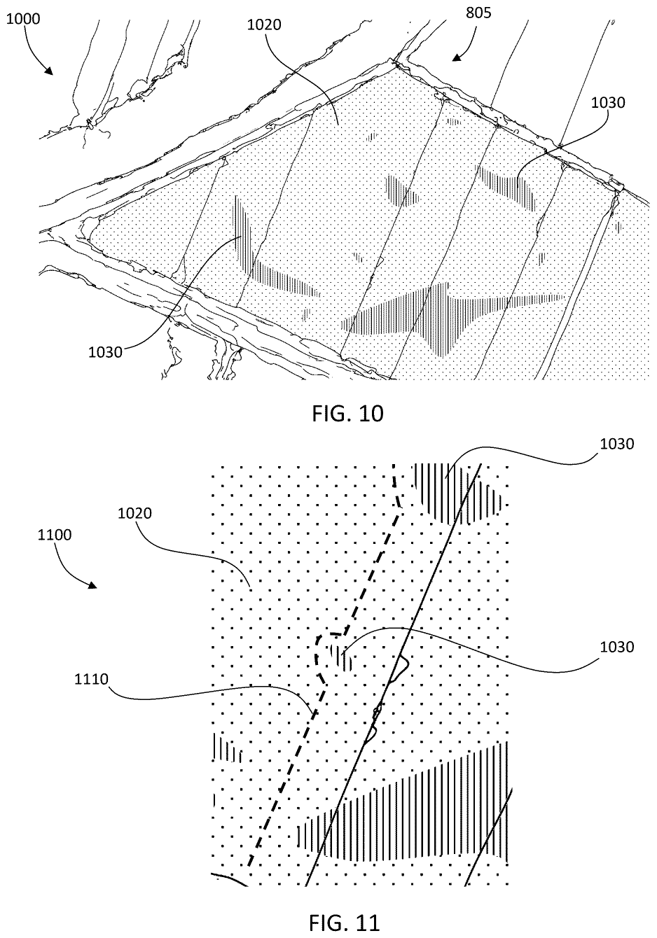

FIG. 10 illustrates a plant health map 1000 comprising the two-dimensional map 800 overlayed with defined portions 1020, 1030, wherein the defined portions may be based on the plant health values of the plant health data. For example, the agricultural field 805 of the plant health map 1000 may comprise a healthy portion 1020 (indicated with a dotted pattern) and an unhealthy portion 1030 (indicated with a hashed pattern). The healthy and unhealthy portions 1020, 1030 may be defined by their relationship to a health threshold. The health threshold may represent a plant health value at which those values at or above the health threshold are considered “healthy” and those values below the health threshold are considered “unhealthy.” For example, those portions of the operating environment associated with plant health values that are greater than or equal to the health threshold may form part of the health portion(s) 1020 of the plant health map 1000, and those portions of the operating environment associated with plant health values that are less than the health threshold may form part of the unhealthy portion(s) 1030.

Each of the two defined portions 1020, 1030 may have an associated operating parameter set. In some embodiments, the plant health map may comprise three or more than three defined portions 1020, 1030, and each of the portions may have an associated operating parameter set, and each of the operating parameter sets may be unique to the type of portion. This may require additional health thresholds. For example, those regions of the operating environment having plant health values lying below a first health threshold may comprise unhealthy portions 1030, those regions of the operating environment having plant health values extending above a second health threshold may comprise healthy portions 1020, and those regions extending between the first and second health thresholds may comprise intermediate portions (not shown). Each of the three portions may then be associated with a distinct set of operating parameters. The autonomous work vehicle 110 may perform the operational task using the set of operating parameters that are distinct to each particular portion when the autonomous work vehicle 110 is operating, for example, within the respective portion of the operating environment.

Alternatively, the autonomous work vehicle 110 may operate based on one or more operating parameters that may be adjusted as a function of the plant health values of the location at which the autonomous work vehicle 110 is operating. For example, the one or more operating parameters may be proportional or inversely proportional to the plant health value of the location of the autonomous work vehicle 110. To illustrate, the autonomous work vehicle 110 may autonomously operate to spray pesticide within the operating environment. The amount of pesticide dispersed by the autonomous work vehicle 110 at the location may be inversely proportional to the plant health value at the location of the autonomous work vehicle 110. In this manner, fewer quantities of pesticide may be used on vegetation that exhibits healthy growth, conserving pesticide use to locations where the pesticide may be needed in larger quantities.

Determining the plant health values and/or generating the plant health map 1000 may be performed after the performance of the operational task. For example, the multispectral sensor data may be stored within the one or more computer-readable media after it is received by the one or more processors 510. The plant health values may then be determined and then used to generate the plant health map 1000. The plant health map 1000 may then be used to operate the autonomous work vehicle 110.

Alternatively, determining the plant health values and/or generating the plant health map 1000 may be performed during the operational task. For example, the plant health map 1000 may be generated progressively over time, for example, as the autonomous tractor 600 follows the path 810. The operation (e.g., the operating parameters) of the autonomous tractor 600 may then be adjusted based on the plant health values within the plant health map 1000. This may ensure that the autonomous tractor 600 operates with a set of operating parameters based on the most current plant health data.

The method 700 may include changing at least one of a default set of operating parameters to a first set of operating parameters for a first portion of the plant health map based on data in the plant health map. For example, a default set of operating parameters may be applied to the operation of the autonomous work vehicle 110 within the operating environment. The default set of operating parameters may be used when plant health data does not indicate that the location at which the autonomous work vehicle 110 operates is associated with plant health values lying below the health threshold. An adjusted set (e.g., a first set) of operating parameters may be defined. The adjusted set of operating parameters may comprise a collection of operating parameters which may be distinct from the collection of operating parameters within the default set of operating parameters. The adjusted set of operating parameters may be used when the plant health data indicates that the location at which the autonomous work vehicle 110 operates is associated with plant health values lying below the health threshold.

The method 700 may then include block 730 in which it is determined whether the autonomous work vehicle 110 is operating within a first portion (such as an unhealthy portion 1030) of the plant health map 1000. When the autonomous work vehicle 110 is found to be operating within the first portion (e.g., unhealthy portion 1030) of the plant health map 1000, the method 700 may proceed to block 735 wherein the autonomous work vehicle 110 may be operated based on the adjusted set (e.g., first set) of operating parameters. When the autonomous work vehicle 110 is found to not be operating within the first portion (e.g., when the autonomous work vehicle 110 is found to be operating within the health portion 1020) of the plant health map 1000, the method 700 may proceed to block 740 wherein the autonomous work vehicle 110 may be operated based on the default set (or a second set) of operating parameters. However, in some embodiments, the autonomous work vehicle 110 may be operated using the adjusted set (e.g., first set) of operating parameters when the autonomous work vehicle 110 is not found to be operating within the first portion. For example, the autonomous work vehicle 110 may be operated using the adjusted set of operating parameters when the autonomous work vehicle 110 is found near the first portion, for example, within a particular distance range.

FIG. 11 shows a close-up view 1100 of the plant health map 1000 of FIG. 10, and further illustrates a portion of an adjusted path 1110 superimposed upon the map 1000. Specifically, the operating parameters may comprise a path 810 of the autonomous work vehicle 110 through the operating environment, and the path 810 may be adjusted based on the plant health values at or near the location of the autonomous work vehicle 110. For example, as the adjusted path 1110 approaches an unhealthy portion 1030 of the operating environment, the adjusted path 1110 may deviate to avoid the unhealthy portion 1030, such that as the autonomous work vehicle 110 follows the adjusted path 1110 the autonomous work vehicle 110 does not drive over the unhealthy portion 1030. This may enable the autonomous work vehicle 110 to prevent or minimize damage to unhealthy portions 1030 of the operating environment and promote plant growth at these portions 1030.

In other embodiments, the path may not be adjusted to cause the autonomous work vehicle 110 to avoid the unhealthy portions 1030. The adjusted set of operating parameters may comprise an adjustment to other operating parameters (e.g., when compared to the default set of operating parameters), such as a velocity or orientation of the autonomous work vehicle 110 at the respective portion of the operating environment. The autonomous work vehicle 110 may perform an action based on the adjusted set (e.g., first set) of operating parameters, for example, by using an attached or coupled implement. For example, the autonomous work vehicle 110 may instruct the implement control system to apply a supplement to a site within the first portion of the plant health map 1000, or to apply an adjusted quantity of the supplement to the site within the first portion. The supplement can comprise one or more of additional water, fertilizer, pesticide, weed killer, soil, or health-promoting spray. In some embodiments, the supplement may comprise additional seed of a desired crop or plant. The operating parameters may include one or more settings of the implement, such that the performance of the implement during the operational task may be based on the plant health values within the plant health map 1000.

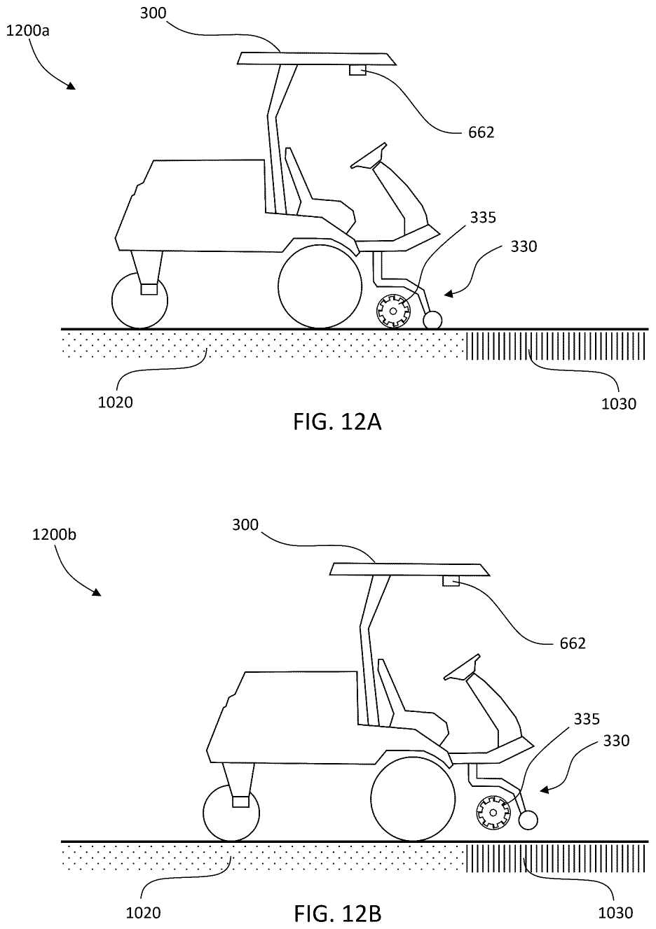

FIGS. 12A and 12B illustrate diagrams 1200a and 1200b, respectively, showing operational adjustment of an autonomous mower 300 based on plant health data. Specifically, the autonomous mower 300 may adjust the height of a mower reel 330 attached to the autonomous mower 300 based on the plant health data. The autonomous mower 300 may be applied to autonomously cut the turf of a golf course or a sod farm.

While the mower 300 is positioned over the healthy portion 1020 of the operating environment, the reel blade 335 of the mower reel 330 may be positioned lower to the surface of the ground, for example, to cut the plants of the operating environment to a desired (or default) height. However, the operating environment may comprise portions where turf grows sparsely, fails to grow, or is in poor condition. The multispectral sensor data received from the multispectral sensor 662 may be used to determine that the plant health value in such locations is below a health threshold, and the region of the operating environment may be defined as an unhealthy portion 1030. When the autonomous work vehicle 110 approaches or begins to drive over the unhealthy portion 1030, the height of the mower reel 330 (and mower blade 335) above the ground may be increased (e.g., to the height within the adjusted set of operating parameters), such that the turf of the unhealthy portion 1030 is not cut to the default height, but at a height which may promote the growth of turf within the unhealthy portion 1030.

Other settings of the implement may be additionally, or alternatively, adjusted based on the plant health data. For example, operating parameters may include the rotor speed, clearance, fan speed, the size of sieve openings, chopper speed, depth of a tillage implement, or other operating parameter of the implement.

Numerous specific details are set forth herein to provide a thorough understanding of the claimed subject matter. However, those skilled in the art will understand that the claimed subject matter may be practiced without these specific details. In other instances, methods, apparatuses or systems that would be known by one of ordinary skill have not been described in detail so as not to obscure claimed subject matter.

Some portions are presented in terms of algorithms or symbolic representations of operations on data bits or binary digital signals stored within a computing system memory, such as a computer memory. These algorithmic descriptions or representations are examples of techniques used by those of ordinary skill in the data processing arts to convey the substance of their work to others skilled in the art. An algorithm is a self-consistent sequence of operations or similar processing leading to a desired result. In this context, operations or processing involves physical manipulation of physical quantities. Typically, although not necessarily, such quantities may take the form of electrical or magnetic signals capable of being stored, transferred, combined, compared or otherwise manipulated. It has proven convenient at times, principally for reasons of common usage, to refer to such signals as bits, data, values, elements, symbols, characters, terms, numbers, numerals or the like. It should be understood, however, that all of these and similar terms are to be associated with appropriate physical quantities and are merely convenient labels. Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed herein are not limited to any particular hardware architecture or configuration. A computing device can include any suitable arrangement of components that provides a result conditioned on one or more inputs. Suitable computing devices include multipurpose microprocessor-based computer systems accessing stored software that programs or configures the computing system from a general-purpose computing apparatus to a specialized computing apparatus implementing one or more embodiments of the present subject matter. Any suitable programming, scripting, or other type of language or combinations of languages may be used to implement the teachings contained herein in software to be used in programming or configuring a computing device.

Embodiments of the methods disclosed herein may be performed in the operation of such computing devices. The order of the blocks presented in the examples above can be varied for example, blocks can be re-ordered, combined, and/or broken into sub-blocks. Certain blocks or processes can be performed in parallel.

Unless otherwise specified, the term “substantially” means within 5% or 10% of the value referred to or within manufacturing tolerances. Unless otherwise specified, the term “about” means within 5% or 10% of the value referred to or within manufacturing tolerances.

The terms “first”, “second”, “third”, etc. are used to distinguish respective elements and are not used to denote a particular order of those elements unless otherwise specified or order is explicitly described or required.

The conjunction “or” is inclusive.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Headings, lists, and numbering included herein are for ease of explanation only and are not meant to be limiting.

While the present subject matter has been described in detail with respect to specific embodiments thereof, it will be appreciated that those skilled in the art, upon attaining an understanding of the foregoing, may readily produce alterations to, variations of, and equivalents to such embodiments. Accordingly, it should be understood that the present disclosure has been presented for purposes of example rather than limitation, and does not preclude inclusion of such modifications, variations and/or additions to the present subject matter as would be readily apparent to one of ordinary skill in the art.

That which is claimed:

1. An autonomous work vehicle comprising:
a steering control system for autonomously controlling a driving direction of the autonomous work vehicle;
a speed control system for autonomously controlling a speed of the autonomous work vehicle;
a geo positioning sensor that records geo positioning data;
a multispectral sensor that records multispectral sensor data;
one or more processors communicatively coupled with the multispectral sensor, the steering control system, the speed control system, and the geo positioning sensor; and
one or more computer-readable media having stored thereon instructions that when executed cause the one or more processors to:
instruct the steering control system and the speed control system to drive the autonomous work vehicle along a path through an operating environment with a default set of operating parameters;
while the autonomous work vehicle is driving along the path, receive and record multispectral sensor data from the multispectral sensor to the one or more computer-readable media;
determine and store in the one or more computer-readable media plant health values for different portions of the operating environment;
generate a plant health map from the plant health values and the geo positioning data, wherein the plant health map comprises a virtual representation of at least a portion of the operating environment;
change at least one of the default set of operating parameters to a first set of operating parameters for a first portion of the plant health map based on data in the plant health map;
operate the autonomous work vehicle based on the first set of operating parameters when operating the autonomous work vehicle is in the first portion of the plant health map; and
operate the autonomous work vehicle based on the default set of operating parameters when operating the autonomous work vehicle outside the first portion of the plant health map.

2. The autonomous work vehicle of claim 1, wherein the autonomous work vehicle further comprises an implement and an implement control system for autonomously controlling operation of the implement, wherein the first set of operating parameters include changing the operation of the implement.

3. The autonomous work vehicle of claim 2, wherein the autonomous work vehicle comprises an autonomous mower, the implement includes a mower reel, and wherein changing at least one of the default set of operating parameters comprises adjusting a height of the mower reel.

4. The autonomous work vehicle of claim 1, wherein changing at least one of the default set of operating parameters includes changing a velocity of the autonomous work vehicle.

5. The autonomous work vehicle of claim 1, wherein generating a plant health map comprises stitching the plant health values and the geo positioning data together to create an orthographic map of the operating environment.

6. The autonomous work vehicle of claim 1, wherein changing at least one of the default set of operating parameters to a first set of operating parameters causes the autonomous vehicle to drive around portions of the operating environment having plant health values below a health threshold.

7. The autonomous work vehicle of claim 1, wherein generating a plant health map from the plant health values and the geo positioning data further comprises projecting pixels onto a two-dimensional map of the operating environment.

8. The autonomous work vehicle of claim 1, wherein generating a plant health map from the plant health values and the geo positioning data further comprises applying a pixel based moving average.

9. The autonomous work vehicle of claim 1, wherein generating a plant health map from the plant health values and the geo positioning data further comprises applying a Bayesian filter to the plant health values.

10. The autonomous work vehicle of claim 1, wherein the multispectral sensor comprises an NDVI camera.

11. The autonomous work vehicle of claim 1, wherein the multispectral sensor comprises a multispectral camera.

12. A method comprising:
instructing a steering control system and a speed control system to drive an autonomous work vehicle along a path through an operating environment with a default set of operating parameters;
receiving multispectral sensor data from a multispectral sensor while the autonomous work vehicle is driving along the path;
recording the multispectral sensor data to one or more computer-readable media;
determining plant health values for different portions of the operating environment;
generating a plant health map from the plant health values and geo positioning data, wherein the plant health map comprises a virtual representation of at least a portion of the operating environment;
changing at least one of the default set of operating parameters to a first set of operating parameters for a first portion of the plant health map based on data in the plant health map;
operating the autonomous work vehicle based on the first set of operating parameters when operating the autonomous work vehicle is in the first portion of the plant health map; and
operating the autonomous work vehicle based on the default set of operating parameters when operating the autonomous work vehicle outside the first portion of the plant health map.

13. The method according to claim 12, wherein changing at least one of the default set of operating parameters includes changing a velocity of the autonomous work vehicle.

14. The method according to claim 12, wherein generating a plant health map comprises stitching the plant health values and the geo positioning data together to create an orthographic map of the operating environment.

15. The method according to claim 12, wherein determining plant health values for different portions of the plant health map comprises using deep learning or other image processing techniques.

16. The method according to claim 12, wherein generating a plant health map from the plant health values and the geo positioning data further comprises projecting pixels onto a two-dimensional map of the operating environment.

17. The method according to claim 12, wherein generating a plant health map from the plant health values and the geo positioning data further comprises applying a pixel based moving average.

18. The method according to claim 12, wherein generating a plant health map from the plant health values and the geo positioning data further comprises applying a Bayesian filter to the plant health values.

19. An autonomous mower comprising:
a steering control system for autonomously controlling a driving direction of the autonomous mower;
a speed control system for autonomously controlling a speed of the autonomous mower;
a mower reel attached to the autonomous mower, wherein the mower reel is configured to cut a surface of an operating environment;
an implement control system for autonomously controlling operation of the mower reel;
a geo positioning sensor that records geo positioning data;
a multispectral sensor that records multispectral sensor data;
one or more processors communicatively coupled with the multispectral sensor, the steering control system, the speed control system, the implement control system, and the geo positioning sensor; and
one or more computer-readable media having stored thereon instructions that when executed cause the one or more processors to:
instruct the steering control system and the speed control system to drive the autonomous mower along a path through an operating environment with a default mower reel height;
while the autonomous mower is driving along the path, receive and record multispectral sensor data from the multispectral sensor to the one or more computer-readable media;
determine and store in the one or more computer-readable media plant health values for different portions of the operating environment;
generate a plant health map from the plant health values and the geo positioning data, wherein the plant health map comprises a virtual representation of at least a portion of the operating environment;
change the default mower reel height to an adjusted mower reel height for a first portion of the plant health map based on data in the plant health map;
operate the mower reel at the adjusted mower reel height when operating the autonomous mower is in the first portion of the plant health map; and
operate the mower reel at the default mower reel height when operating the autonomous mower outside the first portion of the plant health map.
